(19) United States
(12) Patent Application Publication
(10) Pub. No.: US 2025/0151771 A1
(43) Pub. Date: May 15, 2025

(54) CELL-CULTURED MEAT PRODUCTION DEVICE BASED ON MICROFLUIDIC 3D PRINTING TECHNOLOGY AND PROCESS OF PREPARING CELL-CULTURED MEAT BY USE THEREOF

(71) Applicant: NANJING JOES FUTURE FOOD TECHNOLOGY CO., LTD., Jiangsu (CN)

(72) Inventors: Guanghong ZHOU, Jiangsu (CN); Jie WANG, Jiangsu (CN); Xi DING, Jiangsu (CN); Shijie DING, Jiangsu (CN)

(73) Assignee: NANJING JOES FUTURE FOOD TECHNOLOGY CO., LTD., Jiangsu (CN)

(21) Appl. No.: 18/838,612

(22) PCT Filed: Jun. 30, 2023
(86) PCT No.: PCT/CN2023/104677

§ 371 Date: Aug. 15, 2024

(30) Foreign Application Priority Data
Jul. 4, 2022 (CN) 202210781579.4

(51) Int. Cl. A23P 20/20; A23L 13/00; A23P 20/25; B33Y 10/00; B33Y 30/00; B33Y 50/02; B33Y 80/00; C12M 1/00; C12M 1/26; C12M 3/00; C12M 3/06; C12N 5/077

(52) U.S. Cl. CPC A23P 20/20; A23L 13/00; B33Y 10/00; B33Y 30/00; B33Y 50/02; B33Y 80/00; C12M 21/08; C12M 23/16; C12M 29/06; C12M 33/04; C12N 5/0658; A23P 2020/253; C12N 2533/54; C12N 2533/56; C12N 2533/72; C12N 2537/10

(57) ABSTRACT

A cell-cultured meat production device based on microfluidic 3D printing technology includes a printing nozzle, a printing moving system, a loading platform, a sample injection system and a base. The printing moving system is disposed on the base and is composed of a plurality of movable optical axes. The printing nozzle is disposed on a movable optical axis, the sample injection system is connected to the printing nozzle, and the printing nozzle is a micro-fluidic chip. The present invention is based on microfluidic 3D printing, where biological ink is extruded by means of a microfluidic chip to form printed fibers that, under the drive of the printing moving system, are stacked for formation to achieve single-step construction of a piece of three-dimensional tissue having tissue anisotropy, which is used for cell-cultured meat production.

### TECHNICAL FIELD

The present invention belongs to the field of cell-cultured meat, and particularly relates to a cell-cultured meat production device based on microfluidic 3D printing technology and a use thereof.

### DESCRIPTION OF RELATED ART

Meat is a primary source of protein in the diets of people around the world. With the growth of the global population and the rising living standards in developing countries, demands for meat are increasing significantly, putting pressure on the traditional animal husbandry to meet the increasing demands. However, the traditional animal husbandry is resource-intensive, leading to substantial consumption of water and land, and emitting large amounts of greenhouse gases. Furthermore, foodborne diseases and animal welfare issues caused by the traditional animal husbandry should not be underestimated. Therefore, it is of great significance of developing new meat production technologies capable of replacing the traditional animal husbandry.

Cell-cultured meat is a type of novel meat production technology that has emerged from the intersection of cell engineering, tissue engineering, food engineering and other disciplines, and it involves the in vitro large-scale amplification, inducing to differentiate, product collection and food processing of muscle stem cells to obtain meat. Compared with the traditional animal husbandry, the cell-cultured meat can significantly reduce energy consumption, water usage, and greenhouse gas emissions, and eliminate about 99% of land resource use. Since Professor Mark Post in the Netherlands announced the birth of the first cell-cultured meat in 2013, a surge in research and commercialization of the cell-cultured meat was then set off worldwide. Despite considerable research progress, the existing production methods still face significant limitations in muscle fiber formation ability, and meat structure simulation and scalability.

To date, more and more tissue engineering technologies, such as animal and plant protein scaffolds, hydrogels, cell sheet engineering and 3D bio-printing, have been applied to the production of cell-cultured meat. Specifically, the 3D bio-printing is a type of advanced technology that constructs tissue engineering scaffolds and organs according to three-dimensional model instructions by locating and assembling biomaterials or cells upon the principle of additive manufacturing. It has great potential for constructing the cell-cultured meat tissue. At present, the 3D bio-printers on the market are generally expensive, have high maintenance costs, limited printing nozzle structures and simple types, poor flexibility in replacement of the printing nozzles, and face great limitations in batch printing. In addition, no dedicated 3D printing devices are available on the market for the production of cell-cultured meat. Therefore, it is highly anticipated to developing a 3D printing device suitable for the production of cell-cultured meat to achieve low-cost, customized, large-scale, and efficient production of cell-cultured meat.

### SUMMARY

In view of the problems existing in the prior art, the present invention provides a cell-cultured meat production device based on microfluidic 3D printing technology, which effectively solves the problems of high costs, simple function of printing nozzles, poor flexibility, and limited scalability of existing biological 3D printing equipment, and further fills a gap in 3D printing equipment in the field of cell-cultured meat production.

The present invention further provides a process of preparing cell-cultured meat by use of the cell-cultured meat production device based on microfluidic 3D printing technology.

Technical solution: in order to achieve the above objectives, the present invention provides a cell-cultured meat production device based on microfluidic 3D printing technology, including a printing nozzle, a printing moving system, a loading platform, a sample injection system and a base; the printing moving system is disposed on the base and is composed of a plurality of movable optical axes, the printing nozzle is fixed on one of the movable optical axes, and the loading platform is connected to another movable optical axis; and the sample injection system is connected to the printing nozzle, which is a microfluidic chip capable of manipulating, processing, and controlling trace liquid or a sample in a channel.

Preferably, the printing moving system includes an x-axis movable optical axis, a z-axis movable optical axis, and a y-axis movable optical axis, the z-axis and y-axis movable optical axes are fixed on the base, the x-axis movable optical axis is connected to the z-axis movable optical axis, the printing nozzle is fixed on the x-axis movable optical axis, and the loading platform is connected to the y-axis movable optical axis.

Specifically, the printing moving system is composed of moving axes capable of driving the printing nozzle to move in two directions on the x-axis and z-axis movable optical axes, and capable of driving the loading platform to move on the y-axis movable optical axis, and a movable coordinate system configured for the printing moving system can be any one of a Cartesian coordinate system, a triangular coordinate system, a polar coordinate system, or a planar joint coordinate system.

Preferably, the printing nozzle is integrated into the x-axis movable optical axis of the printing moving system, the x-axis movable optical axis is connected to the z-axis movable optical axis, and the printing nozzle is driven by the x-axis and z-axis movable optical axes to move in an xz-plane.

Specifically, the microfluidic chip on the printing moving system can be flexibly replaced to perform integrated printing according to different production demands.

Further, one or more microfluidic chips can be integrated on the printing moving system as the printing nozzles, and a multi-nozzle parallel printing device is further developed to build a multi-nozzle microfluidic 3D printing device.

Further, when a plurality of the microfluidic chips are integrated on the printing moving system, microfluidic chips with a same channel structure or different channel structures can be used.

Further, the microfluidic chip can be integrated on the printing moving system by means of clamping, snap-fitting, plugging, magnetic attraction, tenoning, riveting, threaded connection, or bayonet connection.

Specifically, the loading platform has a detachable structure, and is assembled into the printing moving system, and connected to the y-axis movable optical axis for assembly line printing; and the loading platform is made of copper, aluminum, iron, steel, alloy, glass, ceramic, or carbon fiber plates.

Preferably, the loading platform is combined with the y-axis movable optical axis of the printing moving system and has a detachable structure, and the y-axis movable optical axis drives the loading platform and a printed item formed on the loading platform to move in a y-axis direction.

Further, the loading platform is connected to the y-axis movable optical axis through a snap-fit connection, and can be flexibly detached.

Preferably, the sample injection system is composed of a sample loader, a sample injection pump and a pipe, the sample loader is fixed on the sample injection pump, and the pipe connects an outlet of the sample loader with an inlet of the printing nozzle.

Specifically, the sample injection system include the sample loader, the sample injection pump and the pipe, the sample injection pump is placed on a horizontal table top, the sample loader is fixed on the sample injection pump, one end of the pipe is connected to the outlet of the sample loader, the other end of the pipe is connected to the inlet of the printing nozzle, and a feeding manner of the sample injection system includes piston-type extrusion, pneumatic extrusion or screw-type extrusion.

Preferably, the sample loader is fixed on the sample injection pump through a buckle and can be flexibly disassembled, one end of the pipe is connected with the outlet of the sample loader, and one end of the pipe is connected with the inlet of the printing nozzle.

Preferably, a printing control display system and a data transmission system are embedded and installed in the base; and the data transmission system is connected to the printing control display system wirelessly or through a data cable, and the printing control display system is connected to the printing moving system wirelessly or through a data cable. Specifically, the printing control display system includes a control display screen, which is mainly configured to control printing leveling (zeroing, resetting the printing nozzle, returning to zero, adjusting to a level with a printing platform, and the like), select a printing program, issue a printing instruction and perform a position adjustment of the printing moving system. Functions such as selecting a printing instruction file, adjusting a printing speed, a position of the printing nozzle and a position of the loading platform, and the like, can be implemented by operating on the control display screen.

Specifically, the data transmission system is configured to transmit the printing instruction file into a microfluidic 3D printing device, and a data transmission form of the data transmission system includes USB transmission, memory card transmission or computer transmission. The data transmission system includes an insertion port of a storage device such as a USB and a storage card, and is mainly configured to import the print instruction file into the print control display system of the printer.

Further, the printing control display system and the data transmission system are integrated with the base, an opening is formed on a front of the base and is then embedded in the printing control display system, and an opening is formed on a top of the base and then embedded in an interface of the data transmission system. After being embedded in the base, the printing control display system and the data transmission system are powered together with the base.

Preferably, a front portion and top edges of the rectangular base are opened and embedded in the printing control display system and a plurality of data transmission interfaces as the data transmission system; the base is then placed on the horizontal table top, the y-axis movable optical axis and the z-axis movable optical axis are fixed on the base through bolts, and the x-axis movable optical axis is connected to the z-axis movable optical axis, that is, the printing moving system is successfully assembled; and the printing nozzle is fixed on the x-axis movable optical axis, and the loading platform is connected to the y-axis movable optical axis. The sample injection system is composed of the sample loader, the sample injection pump and a pipe, the sample loader is fixed on the sample injection pump, and the pipe connects an outlet of the sample loader with the inlet of the printing nozzle. In a printing process, the printing nozzle is driven by the x-axis movable optical axis to move on the x-axis, the x-axis movable optical axis can slide on the z-axis movable optical axis, printed tissue on the loading platform is driven by the y-axis movable optical axis to move in the y-axis direction, the movable optical axes in the three directions cooperate with one another to make fibers generated by the printing nozzle stacked for formation in the x-axis, y-axis and z-axis directions. Specifically, the microfluidic chip is a device capable of manipulating, processing, and controlling a trace liquid or sample (with a volume generally of 10⁻⁶-10⁻¹⁵ L) in a channel on a microscopic scale. In the printing process, the printing material is loaded into the sample loader of the sample injection system, the sample loader is fixed on the sample injection pump, the sample loader is connected to the printing nozzle through the pipe, and the material in the sample loader is then pumped into the printing nozzle through the sample injection pump. The printing instruction file is imported into the printing control display system through the data transmission system, and after a target printing instruction file is selected in the printing control display system, the microfluidic 3D printing device is started; and fibers generated thereby is deposited on the loading platform and is stacked for formation in the x-axis, y-axis and z-axis directions according to the printing instruction file, the loading platform is disassembled after completion of the printing, and the printed finished products are collected for subsequent processing operations.

Specifically, materials for making the microfluidic chip include but are not limited to one or more of crystalline silicon, polydimethylsiloxane, quartz, polyphthalamide, polymethyl methacrylate, polycarbonate, polystyrene, epoxy resin, acrylic acid, rubber, and fluoroplastic; and a method for making the microfluidic chip includes glass capillary assembling, machining, etching, or molding.

Specifically, the microfluidic chip can be of a single-channel type, a coaxial nested type, or a multi-channel parallel type.

Preferably, the microfluidic chip is able to, based on different channel structures, generate solid, “shell-core”, hollow, multi-component, spiral, and string-bead fibers for microfluidic 3D printing. The present invention can generate fibers with different structures by building the microfluidic chip with different structures, adjusting and designing a type, a flow rate, and the like, of an introduced fluid.

Preferably, the microfluidic chip is a coaxial nested microfluidic chip.

Further, an inner diameter of an outlet of the microfluidic chip ranges from 200-2000 μm.

The present invention further provides a process of preparing cell-cultured meat by use

of the cell-cultured meat production device based on the microfluidic 3D printing technology, and the process for preparing the cell-cultured meat includes following steps:
step (1) preparing a biological ink required for microfluidic 3D printing for later use;
step (2) importing the printing instruction file into the printing device, loading the biological ink prepared in the step (1) into the sample injection system; connecting the sample injection system to the inlet of the printing nozzle, that is, a channel inlet of the microfluidic chip, squeezing the biological ink into the printing nozzle, that is, the microfluidic chip, through the pipe, selecting the instruction file to be printed after fibers are generated at the outlet of the microfluidic chip, and starting the entire printing device; and the fibers generated at the outlet of the printing nozzle are deposited on the loading platform driven by the optical axes of the printing moving system, and are stacked and shaped according to a printing instruction path;
step (3) disassembling the loading platform, performing crosslinking and curing treatment on the 3D tissue obtained through printing in the step (2), and then transferring the 3D tissue to corresponding culture media for proliferation culture and differentiation culture; and
step (4) harvesting the mature 3D tissue cultured in the step (3), washing to remove the culture medium, and performing food processing to obtain the cell-cultured meat.

Preferably, in the step (2), the printing instruction file is imported into the printing device from the data transmission system, the biological ink prepared in the step (1) is loaded into the sample loader and connected to one end of the pipe, and the sample loader is then fixed on the sample injection pump; the other end of the pipe is connected to the inlet of the printing nozzle, that is, the channel inlet of the microfluidic chip, and the biological ink in the sample loader is squeezed into the microfluidic chip through the pipe by using the sample injection pump; after the fibers are generated at the outlet of the microfluidic chip, the instruction file to be printed is selected in the printing control display system, the microfluidic 3D printing device is started, specifically, after the printing instruction file is clicked on the printing control display system, the printing control display system can provide two instructions, that is, “Print” or “Cancel”, and the printing device is started after “Print” is clicked again. After the printing device is started, the fibers generated at the outlet of the printing nozzle are deposited on the loading platform driven by the x-axis, z-axis and the y-axis movable optical axes of the printing moving system, and are stacked for formation according to the printing instruction path; and
specifically, the biological ink in the step (1) includes a non-adhesive cell material solution and a hydrogel solution containing seed cells; and the hydrogel solution containing seed cells contains 30%-70% biological material, 0.01%-1% crosslinking agent in a volume ratio, and balance is a basal medium containing a calcium salt and containing the seed cells of 5×10⁶-5×10⁸/mL.

Preferably, the non-adhesive cell material solution in the biological ink is any one or more of sodium alginate, chitosan, pectin, carrageenan, and gellan gum; and a concentration of the non-adhesive cell material solution is 10-50 mg/mL.

Specifically, the seed cells in the biological ink are derived from any one or more of a pig, cattle, sheep, a chicken, a duck, a rabbit, a fish, a shrimp, and a crab; and the seed cells are one or more of muscle stem cells, myoblasts, myosatellite cells, muscle precursor cells, bone marrow-derived mesenchymal stem cells, adipose-derived mesenchymal stem cells, induced pluripotent stem cells, cardiomyocytes, adipose stem cells, adipose precursor cells, bone marrow-derived adipose cells, fibroblasts, smooth muscle cells, vascular endothelial cells, epithelial cells, neural stem cells, glial cells, osteoblasts, chondrocytes, liver stem cells, hematopoietic stem cells, stromal cells, embryonic stem cells, or bone marrow stem cells.

Preferably, the seed cells are derived from any one or more of the pig, the cattle, the sheep, the chicken, or the duck; and the seed cells include but are not limited to the muscle stem cells, the myoblasts, the myosatellite cells, or the muscle precursor cells.

Specifically, the biological material in the step (1) is one or more of collagen, recombinant collagen, gelatin, matrigel, hyaluronic acid, silk protein, elastin, spider silk protein, fibrin, fibrinogen, silk fibroin, laminin, fibronectin, integrin, cadherin, nestin, decellularized extracellular matrix, chondroitin sulfate, heparin, keratan sulfate, dermatan sulfate, heparan sulfate, keratin, keratin sulfate, cellulose, polymerase, carboxymethyl cellulose, polylactic acid, polyvinyl alcohol, lecithin, nanocellulose, soy protein, pea protein, gluten protein, rice protein, peanut protein, yeast protein, fungal protein, wheat protein, potato protein, corn protein, chickpea protein, mung bean protein, seaweed protein, almond protein, or quinoa protein, and other materials with biocompatibility and capable of providing adhesion sites for the seed cells.

Preferably, the biological material is one or more of the collagen, the recombinant collagen, the gelatin, the matrigel, the hyaluronic acid, or the silk protein. Specifically, the basal medium used in the biological ink includes but is not limited to one or more of F-10, DMEM, MEM, F-12, DMEM/F-12, DMEM/F-12 GlutamMAX™, F-12K, RPMI 1640, IMDM, L-15,199, MCDB 131, LHC, or McCoy's 5A.

Preferably, the basal medium is one or more of the F-10, the DMEM, the MEM, the F-12 or the DMEM/F-12. Specifically, the crosslinking agent used in the biological ink includes but is not limited to any one or more of pH regulators, such as NaOH, KOH, NaHCO₃, HEPES balanced salt solution, EBSS balanced salt solution, HBSS balanced salt solution, PBS, and DPBS, transglutaminase, tyrosinase, laccase, lysyl oxidase, polyphenol oxidase, catalase, thrombin or genipin.

Preferably, the crosslinking agent includes one or more of NaOH, KOH, or NaHCO₃.

Preferably, the hydrogel solution includes the biological material, the crosslinking agent, and the basal medium containing a calcium salt and containing the seed cells; each 1 mL of the hydrogel solution contains 290-699 μL of 4-8 mg/mL biological material, 1-10 μL of 1-2 mol/L crosslinking agent, and 300-700 μL of the basal medium containing 15-25 mg/mL of the calcium salt and 1×10⁷-1×10⁸ of the seed cells; the biological material is one or more of the collagen, the recombinant collagen, the gelatin, the matrigel, the hyaluronic acid, or the silk protein; the crosslinking agent includes one or more of NaOH, KOH, or NaHCO₃; the calcium salt is one or more of calcium chloride, calcium carbonate, calcium sulfate and calcium nitrate; the basal medium is one or more of the F-10, the DMEM, the MEM, the F-12 or the DMEM/F-12; and the seed cells are the muscle stem cells, the myoblasts, the myosatellite cells, or the muscle precursor cell of the pig, the sheep, the chicken or the duck.

More preferably, the hydrogel solution includes the biological material, the crosslinking agent, and the basal medium containing a calcium salt and containing the seed cells; the biological material in each 1 mL of the hydrogel solution includes 150-650 μL of 6 mg/mL collagen and 40-149 μL of the matrigel, the crosslinking agent is 1-10 μL of 1 mol/L alkali solution, and the basal medium containing the calcium salt and the seed cells is 300-700 μL of a DMEM solution containing 15-25 mg/mL of the calcium salt to resuspend 1.5×10⁶-1.5×10⁸ muscle stem cells.

Further, the seed cells are porcine muscle stem cells; the hydrogel solution includes the biological material, the crosslinking agent, and the basal medium containing a calcium salt and containing the seed cells; the biological material in each 1 mL of the hydrogel solution includes 600 μL of 6 mg/mL collagen and 97 μL of the matrigel, the crosslinking agent is 3 μL of 1 mol/L NaOH, and the basal medium containing the calcium salt and the seed cells is 300 μL of the DMEM solution containing 20 mg/mL of the calcium salt to resuspend 1.5×10⁷ muscle stem cells.

Preferably, the crosslinking agent includes one or more of NaOH, KOH, or NaHCO₃.

Preferably, the hydrogel solution containing seed cells includes the biological material, the crosslinking agent, and the basal medium containing a calcium salt and containing the seed cells; each 1 mL of the hydrogel solution contains 290-699 μL of 4-8 mg/mL biological material, 1-10 μL of 1-2 mol/L crosslinking agent, and 300-700 μL of the basal medium containing 15-25 mg/mL of the calcium salt and 1×10⁷-1×10⁸ of the seed cells; the biological material is one or more of the collagen, the recombinant collagen, the gelatin, the matrigel, the hyaluronic acid, or the silk protein; the crosslinking agent includes one or more of NaOH, KOH, or NaHCO₃; the calcium salt is one or more of calcium chloride, calcium carbonate, calcium sulfate and calcium nitrate; the basal medium is one or more of the F-10, the DMEM, the MEM, the F-12 or the DMEM/F-12; and the seed cells are the muscle stem cells, the myoblasts, the myosatellite cells, or the muscle precursor cell of the pig, the sheep, the chicken or the duck.

More preferably, the hydrogel solution containing seed cells includes the biological material, the crosslinking agent, and the basal medium containing a calcium salt and containing the seed cells; the biological material in each 1 mL of the hydrogel solution includes 150-650 μL of 6 mg/mL collagen and 40-149 μL of the matrigel, the crosslinking agent is 1-10 μL of 1 mol/L alkali solution, and the basal medium containing the calcium salt and the seed cells is 300-700 μL of a DMEM solution containing 15-25 mg/mL of the calcium salt to resuspend 1.5×10⁶-1.5×10⁸ muscle stem cells.

Further, the seed cells are porcine muscle stem cells; the hydrogel solution containing seed cells includes the biological material, the crosslinking agent, and the basal medium containing a calcium salt and containing the seed cells; the biological material in each 1 mL of the hydrogel solution includes 600 μL of 6 mg/mL collagen and 97 μL of the volume ratio, the crosslinking agent is 3 μL of 1 mol/L NaOH, and the basal medium containing the calcium salt and the seed cells is 300 μL of the DMEM solution containing 20 mg/mL of the calcium salt to resuspend 1.5×10⁷ muscle stem cells.

Specifically, further, a moving speed of each movable optical axis in the printing moving system in the step (2) ranges from 0.5-50 mm/s.

The crosslinking and curing treatment in the step (3) includes but is not limited to one

or more of temperature-induced crosslinking, electrostatic interaction crosslinking, ion crosslinking, and enzyme crosslinking.

Specifically, the basal medium used in preparing the culture medium in the step (3) includes but is not limited to F-10, DMEM, MEM, F-12, DMEM/F-12, DMEM/F-12GlutamMAX™, F-12K, RPMI 1640, IMDM, L-15, 199, MCDB 131, LHC, or McCoy's 5A.

In the step (3), the culture medium for proliferation culture includes 79-89% basal medium, 10-20% fetal bovine serum, and 1% penicillin-streptomycin in the volume ratio, and 1-10 ng/ml alkalic fibroblast growth factor is added into the solution; and the culture medium for differentiation culture includes 94-97% basal medium, 2-5% horse serum and 1% penicillin-streptomycin in the volume ratio.

Specifically, the food processing in the step (4) includes preprocessing and cooking, and the preprocessing includes cleaning, seasoning, color enhancement, modeling, sensory quality modification, and the like, and the cooking includes frying, deep frying, boiling, steaming, baking, and the like.

The present invention adopts the 3D stacking formation method of fiber material as a design principle, the main idea is to modify the conventional extrusion-type 3D printing device, and the microfluidic chip is configured to replace the printing nozzle of the extrusion-type 3D printing device, such that t the plastic fiber material originally used for stacking formation is changed into the biomimetic fiber loaded with cells, and the 3D tissue is further constructed. The present invention effectively solves the problems of high costs, simple function of printing nozzles, poor flexibility, and limited scalability of existing biological 3D printing equipment, and fills a gap in 3D printing equipment in the field of cell-cultured meat production.

In the present invention, the microfluidic chip is configured to replace the printing nozzle of the extrusion-type 3D printing device, and the microfluidic 3D printing device is accordingly developed; and unlike the integrated printing nozzle on various 3D printing devices available on the market, the microfluidic chip used as the printing nozzle can be flexibly designed in the type and the channel structure, a number of the microfluidic chips used for printing can be flexibly increased, and the material for building the microfluidic chips are low in cost, and easy to obtain.

In the present invention, muscle fibers constitute the most basic component units of skeletal muscle tissue, and unlimited muscle fibers are wrapped by connective tissue membranes to form a large skeletal muscle tissue; and the cell-cultured meat production device based on microfluidic 3D printing technology provided in the present invention takes the biomimetic fiber as a basic unit, and the 3D tissue is formed after stacking in layer by layer, and the 3D tissue to the biomimetic fiber likes muscle fiber to the muscle, such that the biomimetic fiber has good biomimetic properties. Compared with the biomimetic fiber, the growth of the seed cells in the 3D tissue further simulates their growth in the natural tissue, and a stacking direction of the biomimetic fiber can be adjusted through the printing instruction path, which is conducive to reproducing the tissue anisotropy of the skeletal muscle tissue in vitro.

In the present invention, the industrial 3D printing device nozzle is improved, where the printing device nozzle is replaced by the low-cost microfluidic chip with a customizable structure, and the number and form of the microfluidic chip used as the printing nozzle of the printing device can be flexibly changed by designing the connection mode between the microfluidic chip and the microfluidic 3D printing device. Based on 3D printing technology, a printing precision (a minimum size of the extruded fiber) of the printing device in the present invention depends on a size of the outlet of the microfluidic chip, an inner diameter of the outlet of the microfluidic chip ranges from 200-2000 μm, and the minimum size thereof has reached 0.2 mm of a standard precision of the 3D printing slice software. In addition, cell-cultured meat products printed in different batches have almost no change in size, and can accurately reproduce the printing path of the 3D model after slicing in the slicing software.

In the 3D printing process, the present invention adopts the principle of a basic unit of natural skeletal muscle tissue-muscle fiber structure, an appropriate fiber carrier is given to the seed cells to ensure that good effects are achieved in the efficient production of the cell-cultured meat; and the microfluidic technology is adopted in the production of the cell-cultured meat for the first time, such that a trace liquid can be manipulated in the micro-size channel. The present invention takes biomimetic muscle fibers as design principles and provides a biomimetic fiber carrier for cell-cultured meat production based on 3D printing combined with microfluidics, which has not been reported in the field for producing the cell-cultured meat. The present invention adopts microfluidic 3D printing technology to prepare biomimetic fibers, which are then used for cell-cultured meat production. The fiber preparation process is continuous and rapid, and the fibers prepared therefrom exhibit good biomimetic properties, greatly improving the directional growth and differentiation abilities of the seed cells within the fibers. The present invention prepares biomimetic fibers with a “core-shell” structure based on the microfluidic technology. The seed cells are encapsulated within the shell made of non-adhesive cell material, featuring highly orientation and fused growth under the spatial constraints of the shell, the in vitro myogenic differentiation ability is also greatly improved, and the production efficiency is accordingly improved. Furthermore, the prepared fibers are very similar to natural skeletal muscle fibers both in shape and physiological characteristics, therefore, the fibers prepared using the microfluidic technology exhibit good biomimetic properties.

In the 3D printing process of the present invention, a coaxial nested microfluidic chip is first designed and built; materials are then prepared, the biomimetic fibers are then stably generated and cultured by adjusting the parameters such as inlet sequence and flow rate of the internal and external phases, and the cultured biomimetic fibers are finally organized, integrated and processed to obtain the cell-cultured meat products. Compared with the common production methods that use scaffolds or bulk hydrogel carriers, the seed cells are encapsulated seed cells are encapsulated within the shell made of non-adhesive cell material, featuring highly orientation and fused growth under the spatial constraints of the shell, the in vitro myogenic differentiation ability is also greatly improved, and the production efficiency is accordingly improved.

In the present invention, cell culture is performed in the biomimetic fibers with a specific “core-shell” structure formed by 3D printing, which effectively improves the differentiation ability of primary cells of the livestock and poultry in the microfibers, such as muscle stem cells, therefore, the synthesis of muscle-related proteins is increased, and mature muscle fibers are further formed, thereby improving the efficiency of cell-cultured meat production. After 2 days of proliferation and 14 days of differentiation culture, the microfibers prepared by the present invention exhibit spontaneous contraction and beating, which results from the migration and fusion of primary myoblasts in the specific core wrapped by the calcium alginate shell to form multinucleated myotubes, and the multinucleated myotubes further differentiate and highly express myosin, thereby forming mature muscle fibers with certain physiological functions.

After proliferation and differentiation culture, the biomimetic fibers formed by the present invention, after 7 days of differentiation, show a compact microfiber structure through the observation of the scanning electron microscopy (SEM) and H&E tissue staining, and obvious myotube structure could be observed on the surface thereof; and the tissue sections also show staining characteristics close to pork fibers, closely resembling the pork fibers on the whole; and after 14 days of differentiation, spontaneous contraction and beating are observed under bright field microscopy, which proves a successful transition from cells to mature tissue under in vitro culture conditions, indicating that the microfibers composed of the seed cells have fully matured to form muscle fibers with contraction function similar to natural muscle fibers. The prevent invention can culture a muscle fiber in vitro, and collecting and assembling the in vitro cultured muscle fibers could theoretically give rise to a block of cultured meat composed of the muscle fibers.

Beneficial effects: compared with the prior art, the present invention has the following advantages:
(1) The present invention provides a cell-cultured meat production device based on microfluidic 3D printing technology, which is used for the cell-cultured meat production, and has the advantages of convenient operation, rapid molding, high degree of mechanization, low cost of required equipment and devices, high precision and stable quality of the printed finished products.
(2) The 3D tissue prepared using microfluidic 3D printing in the present invention exhibits good tissue anisotropy, further simulating the growth of seed cells in natural tissues, and having excellent biomimetic properties.
(3) In the cell-cultured meat prepared by the present invention, the seed cells are directionally arranged within the fibrous basic structure of printed 3D tissue and are further migrated and fused, thereby enhancing the differentiation and maturation of seed cells, and further improving the efficiency of cell-cultured meat production.
(4) The cell-cultured meat using microfluidic 3D printing technology according to the present invention is highly programmable, and features of the cell-cultured meat, such as size and shape, can be adjusted at will in the modeling software, enabling the further development of various types of cell-cultured meat products and improving market acceptance.
(5) The microfluidic 3D printing device developed in the prevent invention can be used as the microfluidic chip of the print head, which can be flexibly replaced, and the chip channel structure can also be customized according to the production needs, so as to produce cell-cultured meat with various structures and components, which has significant potential for diversification and personalized customization of the cell-cultured meat products.
(6) The microfluidic 3D printing device can integrate a plurality of the microfluidic chips on its printing moving system as needed. The loading platform can also be combined with a transmission system to achieve multi-nozzle assembly line printing, which is conducive to promoting the scaling and industrialization of cell-cultured meat.

### BRIEF DESCRIPTION OF THE DRAWINGS

FIG. 1 is a schematic diagram of a cell-cultured meat production device based on microfluidic 3D printing technology according to the present invention.

FIG. 2 shows a structural schematic diagram and a photo of a microfluidic chip channel structure of a cell-cultured meat production device based on microfluidic 3D printing technology according to the present invention, with a scale of 200 μm.

FIG. 3 shows physical diagrams of cell-cultured meat produced by microfluidic 3D printing technology according to the present invention, where (a) is a physical diagram of a printing process; (b) is a physical diagram of a finished product, with a scale of 1000 μm.

FIG. 4 shows printed finished products of different shapes constructed by microfluidic 3D printing technology according to the present invention, where (a) is triangular, (b) is hexagonal, (c) is circular, (d) is heart shaped, with a scale of 1000 μm.

FIG. 5 shows physical diagrams of cell-cultured meat produced by multi-nozzle

microfluidic 3D printing technology according to the present invention, where (a) is a physical diagram of a printing process; (b) is a physical diagram of a finished product, with a scale of 1000 μm.

FIG. 6 is microscope bright field images of a culture process of cell-cultured meat produced by microfluidic 3D printing technology according to the present invention, where (a) is a 3D tissue microscope bright field image (4 h after completion of the printing), and (b) is a 3D tissue microscope bright field image after 2 d of culture, with a scale of 400 μm.

FIG. 7 shows tissue immunofluorescence staining images of cell-cultured meat produced by microfluidic 3D printing technology according to the present invention after culture and maturation, where (a) is a cell nucleus, (b) is cytoskeletal protein, (c) is myosin, and (d) is a fusion image, with a scale of 100 μm.

FIG. 8 is an imaging diagram of protein composition of cell-cultured meat produced by microfluidic 3D printing technology according to the present invention and polyacrylamide gel electrophoresis gel of commercially available pork.

FIG. 9 is a diagram of comparison in appearance between a finished product of cell-cultured meat produced by microfluidic 3D printing technology according to the present invention after food processing and commercially available pork, where the left piece is the commercially available pork, and the right piece is the cell-cultured meat, with a scale of 2000 μm.

FIG. 10 shows data diagrams of comparison in texture characteristics between a finished product of cell-cultured meat produced by microfluidic 3D printing technology according to the present invention after food processing and commercially available pork, where (a) is hardness, (b) is elasticity, (c) is chewiness, and (d) is cohesiveness.

FIG. 11 is a graph of changes in expression levels of cell differentiation-related genes and proteins based on qPCR and Western Blot during the differentiation culture of biomimetic fibers, where (a) is MyoG gene, (b) is MyHC-2a gene, (c) is MyHC-slow gene, (d) shows a Western Blot band diagram of related proteins, (e) shows a gray value analysis of a MyoG protein band, and (f) shows a gray value analysis of a Myosin protein band.

FIG. 12 shows immunofluorescence staining images and statistical diagrams of biomimetic fibers after maturation, where (a) is an immunofluorescence staining image, specifically, i shows a cell nucleus, ii shows a cytoskeletal protein, iii shows myosin, and iv shows a fusion image, with a scale of 100 μm; (b) is a statistical analysis diagram of directionality of the cytoskeletal protein, (c) is a statistical analysis diagram of a roundness and an aspect ratio of the cell nucleus, and (d) is a statistical analysis diagram of a positive cell of the myosin and a myotube region.

### DESCRIPTION OF THE EMBODIMENTS

The present invention will be further described with reference to the accompanying drawings and the embodiments.

Raw materials and reagents used in the examples are commercially available. Specifically, seed cells are obtained by using existing conventional separation and purification methods or directly commercially available.

### Example 1

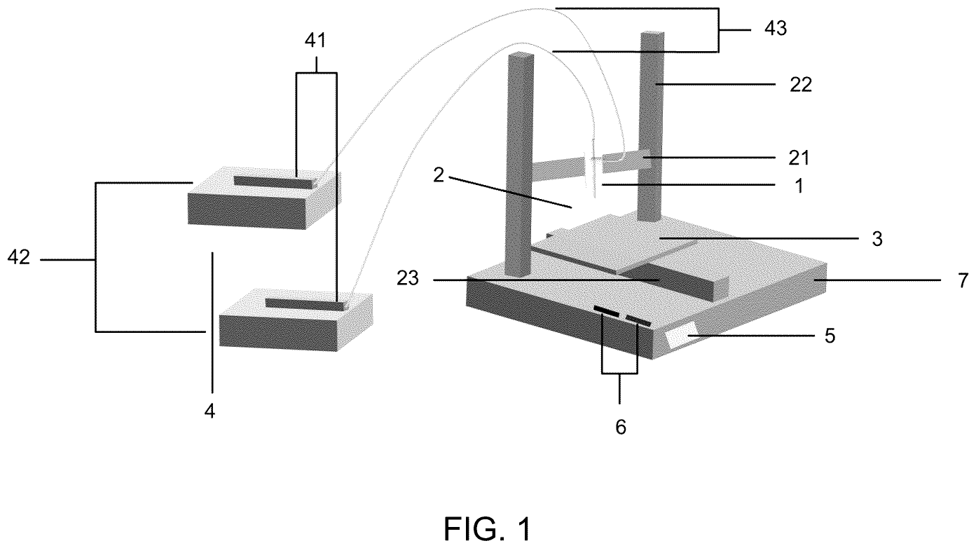

A schematic diagram of a cell-cultured meat production device based on microfluidic 3D printing technology is shown in FIG. 1:
the device includes a printing nozzle 1, a printing moving system 2, a loading platform 3, a sample injection system 4, a printing control display system 5, a data transmission system 6, and a base 7.

The printing nozzle 1 is a microfluidic chip, which is clamped and fixed on an x-axis movable optical axis 21 in the printing moving system 2, and is driven by the x-axis movable optical axis 21 to move in an x-axis direction; and the x-axis movable optical axis 21 is connected with to a z-axis movable optical axis 22 through a bolt, and is driven by the z-axis movable optical axis 22 to move in a z-axis direction. The loading platform 3 is installed on a y-axis movable optical axis 23 of the printing moving system 2 through a buckle, the y-axis movable optical axis 23 drives the loading platform 3 and a printed product formed on the loading platform 3 to move in a y-axis direction, and the loading platform 3 is detachable to collect a sample, and the movable optical axis, the loading platform and the base are generally made of aluminum alloy.

The sample injection system 4 includes a sample loader 41, a sample injection pump 42 and a pipe 43, the sample loader 41 is fixed on the sample injection pump 42 and can be flexibly disassembled for loading printing material, one end of the pipe 43 is connected to an outlet of the sample loader 41, and the other end thereof is connected to an inlet of the printing nozzle 1. The present invention has no special limitations on the type of the sample injection pump 42, and a syringe pump well known to those skilled in the art applicable to a syringe can be used, and in this example, the sample injection pump 42 is a Longer Pump LSP 01-1A micro-injection pump. The sample loader 41 adopts a syringe well known to those skilled in the art, and has no special limitations on brand, type and size of the syringe; and the pipe 43 adopts a polyethylene plastic pipe well known to those skilled in the art, and has no special limitation on brand, type and size of the polyethylene plastic pipe, and in this example, an outer diameter of the polyethylene plastic pipe is 1.3 mm, and an inner diameter thereof is 0.9 mm.

The printing control display system 5 and the data transmission system 6 are integrated with the base 7, an opening is formed on a front of the base 7 and is then embedded in the printing control display system 5, and an opening is formed on a top of the base and then embedded in an interface of the data transmission system 6, the data transmission system 6 is connected to the printing control display system 5 through a data cable, and the printing control display system 5 is connected to the printing moving system 2 through a data cable. Specifically, a front portion and top edges of the rectangular base 7 are opened to be connected to the printing control display system 5 and the data transmission system 6, the printing control display system 5 is mainly configured to control printing leveling, select a printing program, issue a printing instruction and perform a position adjustment of the printing moving system 2; the data transmission system 6 is configured to transmit the printing instruction file into a microfluidic 3D printing device; and a data transmission form of the data transmission system 6 includes USB transmission, memory card transmission or computer transmission. The base 7 is placed on a horizontal table top, the y-axis movable optical axis 23 and the z-axis movable optical axis 22 are fixed on the base 7 through bolts, and the x-axis movable optical axis 21 is connected to the z-axis movable optical axis 22, that is, the printing moving system 2 is successfully assembled.

The microfluidic chip can be a single-channel device for printing solid fiber. An outlet of one glass capillary is drawn into a size with an outer diameter of 200 μm and an inner diameter of 100 μm, and the drawn glass capillary is glued onto a glass sheet with AB glue to build a single-channel microfluidic chip. The microfluidic chip can also be a coaxial nested device for printing hollow fiber, “shell-core” fiber, spiral fiber, string-bead fiber, and the like. The present invention has no special limitations on a number of channels of the pipe in the microfluidic chip, and the number of channels of the pipe can be two, three or four.

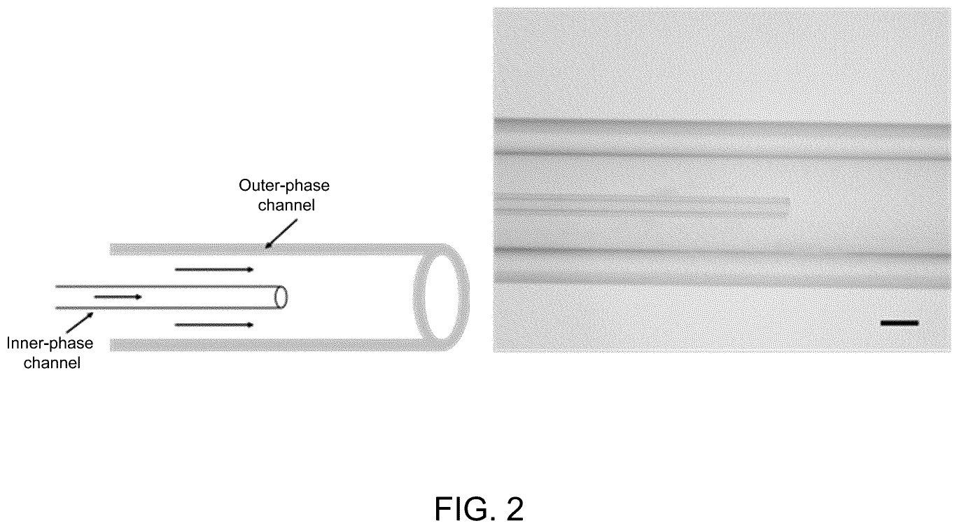

In this example, a coaxial nested microfluidic chip is specifically used, the coaxial nested microfluidic chip includes an inner-phase glass capillary for introducing an inner-phase solution, and an outer-phase glass capillary for introducing an outer-phase solution. The coaxial nested microfluidic chip is composed of the glass capillary, a dispensing needle and a glass sheet, the glass capillary is cylindrical, and the dispensing needle is a 20 G dispensing needle; the present invention has no special limitations on type and size of the glass sheet, and the glass sheet is a commercially available glass slide with a thickness of 1 mm; and the glass slide has a length of 30 mm and a width of 25 mm. A specific method is as follows: selecting a cylindrical glass capillary with an inner diameter of 580 μm and an outer diameter of 1000 μm, and pulling an outlet of the glass capillary into a size with an inner diameter of about 80 μm to serve as an inner-phase channel; and then selecting one more cylindrical glass capillary with an inner diameter of 580 μm and an outer diameter of 1000 μm, and pulling an outlet of the glass capillary into a size with an inner diameter of about 200 μm to serve as an outer-phase channel. The outer-phase channel is fixed to a central position on the glass slide, a drawing end of the inner-phase channel is inserted from one end of the outer-phase channel to ensure that the two-phase channels do not block each other, and the outer-phase channel and the inner-phase channel are adjusted to a same axis under a stereo microscope to fix the two tubes; and the 20 G dispensing needle is fixed at a joint of the two-phase channels, and assembly thereof is completed after being glued with the AB glue, and a structural schematic diagram and a micrograph are shown in FIG. 2 as a microfluidic chip for printing 3D tissue in Example 3.

The printing nozzle 1 in this example is configured to be composed of a plurality of the microfluidic chips to facilitate multi-nozzle microfluidic 3D printing. In addition, materials for making the microfluidic chip can be replaced by crystalline silicon, polydimethylsiloxane, quartz, polyphthalamide, polymethyl methacrylate, polycarbonate, polystyrene, epoxy resin, acrylic acid, rubber, and fluoroplastic.

### Example 2

### Preparation of Microfluidic 3D Printing Material

Preparation of microfluidic outer-phase fluid: an appropriate amount of sodium alginate

powder was taken and placed in an ultra-clean workbench for ultraviolet irradiation sterilization, and kept overnight. 20 mL of sterile water was taken into a centrifuge tube by using a pipette, 0.6 g of sodium alginate powder was weighed in the ultra-clean workbench by using an electronic balance and poured the same into the centrifuge tube to obtain a mixture, the mixture was stirred and mixed evenly by a vortex mixer, the centrifuge tube was then placed into a 37° C. thermostat water bath and incubated for 15 min, the centrifuge tube was taken out and was subjected to vortex again, the above operation was repeated for 3-5 times until the sodium alginate powder was completely dissolved, and a 30 mg/mL sodium alginate solution was then obtained and was centrifuged at 3000×g for 5 min to remove bubbles in the sodium alginate solution for later use (for printing 3D tissue in Example 3).

Preparation of inner-phase microfluidic fluid: 0.1 g of calcium chloride was weighed and placed in a centrifuge tube, 5 mL of DMEM basal medium containing phenol red (C11995500CP, Gibco) was added to the centrifuge tube for dissolution to obtain a DMEM solution containing 20 mg/mL of calcium chloride, and the DMEM solution was filtered with a 0.22 μm filter membrane for sterilization and then stored on an ice for later use; 0.2 g of NaOH was weighed and placed in a centrifuge tube, 5 mL of ultrapure water was added to the centrifuge tube for dissolution to obtain a 1 mol/L NaOH solution, the NaOH solution was filtered with a 0.22 μm filter membrane for sterilization and then stored on an ice for later use.

1 mL of an inner-phase fluid system was taken as an example, cell suspension containing 1.5×10⁷ porcine muscle stem cells was taken and placed in a centrifuge tube, and centrifuged at 300×g for 5 min, supernatant thereof was removed to obtain cell precipitate, and the cell precipitate was placed on ice for later use. The 1.5×10⁷ porcine muscle stem cells were resuspended with 300 μL of the DMEM solution containing 20 mg/mL calcium chloride, 600 μL of 6 mg/mL collagen (collagen derived from bovine skin, Sigma, Model C2124) was added to the cell suspension, which was transferred as a whole to the 2 mL centrifuge tube containing 3 μL of 1 mol/L NaOH solution, 97 μL of matrigel (standard Matrigel, Corning Incorporated) was added and then gently blown and mixed evenly with 1 mL of pipette tip to obtain a hydrogel solution, and finally, the obtained hydrogel solution was placed and stored on an ice for later use (for printing 3D tissue in Example 3).

In addition, the same preparation method described above can be adopted, except that: the non-adhesive cell material solution is chitosan and has a concentration of 10 mg/mL; the hydrogel solution contains gelatin with a volume ratio of 30%, a genipin solution with a volume ratio of 1%, and F-10 medium containing calcium chloride with a volume ratio of 69% and containing 5×10⁶/mL bovine muscle stem cells.

Alternatively, differences lie in that: the non-adhesive cell material solution is pectin with a concentration of 50 mg/mL; the hydrogel solution contains hyaluronic acid with a volume ratio of 70%, a carbodiimide solution with a volume ratio of 1%, and MEM medium containing calcium chloride with a volume ratio of 29% and containing 5×10⁸/mL chicken muscle stem cells.

Alternatively, differences lie in that: the non-adhesive cell material solution is carrageenan with a concentration of 25 mg/mL; the hydrogel solution contains fibrinogen with a volume ratio of 50%, a thrombin solution with a volume ratio of 0.5%, and DMEM/F-12containing calcium chloride with a volume ratio of 49.5% and containing 5×10⁷/mL sheep muscle stem cells.

With the specific 3D printing technology of the present invention, effects of the present invention can be achieved by culturing a certain amount of porcine, bovine, sheep, chicken, duck and other muscle stem cells.

### Example 3

### Preparation of 3D Tissue

A printing model was established by using Auto CAD 2021, dimensions of the printing model were 15 mm×20 mm×2 mm, and the printing model was exported as an.stl model file format; the.stl model file was imported in Cura Slicing, a printing interval was set to be 0.7 mm, a printing speed was 5 mm/s, and the slicing program was run to obtain a G-code printing instruction file; and the G-code printing instruction file was saved to a mobile disk, and then imported into the printing control display system 5 of a microfluidic 3D printing device through the data transmission system 6 for later use.

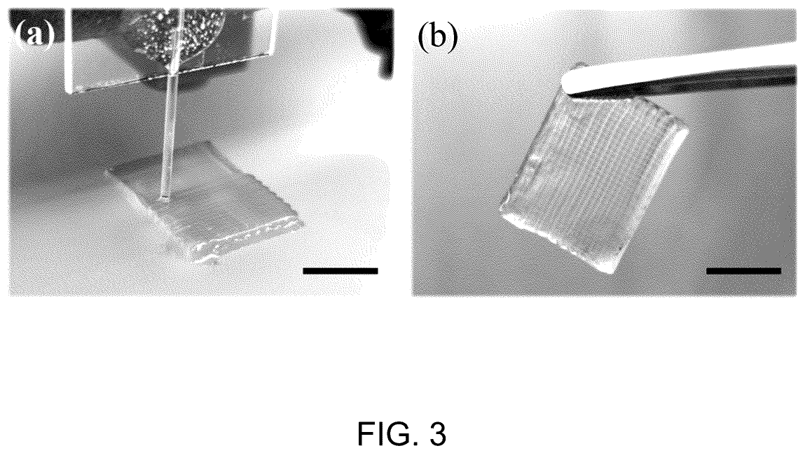

The coaxial nested microfluidic chip constructed in Example 1 was used, the sodium alginate solution prepared in Example 2 was added to one 5 mL syringe, one end of a segment of polyethylene plastic tube was connected to a needle of the syringe, and the other end thereof was connected to an outer-phase inlet of the microfluidic chip; and the hydrogel solution containing porcine muscle stem cells prepared in Example 2 was added to another 2 mL syringe, one end of a polyethylene plastic tube was connected to a needle of the syringe, and the other thereof was connected to an inner-phase inlet. Then, the syringes containing the two-phase fluids were respectively fixed on two pumps, a flow rate of the inner-phase hydrogel solution was adjusted to be 1.7 mL/h, and a concentration of the outer-phase sodium alginate solution was 1.8 mL/h. Driven by the pumps, inner-phase and outer-phase printing materials were introduced into the microfluidic chip through the polyethylene plastic tubes, such that that the two-phase fluids formed a stable laminar flow structure in the device to form biomimetic fiber with a “shell-core” structure from an outlet of the microfluidic chip (that is, an outlet of the outer-phase channel). After the fiber was generated at an outlet of the printing nozzle (the outlet of the outer-phase channel), the printing program was selected by the printing control display system 5 and the microfluidic 3D printing device was started, the microfluidic chip of the microfluidic 3D printing device was then driven by the printing moving system 2 to move on the x axis and the z axis, the printing sample was driven by the loading platform to move on the y axis, a moving speed each optical axis was 5 mm/s, fibers generated therefrom were deposited on the loading platform and stacked according to a G-code printing instruction path, and the 3D tissue was obtained after printing, a 10 mg/mL calcium chloride solution was prepared, sterilized and used as an ionic crosslinking agent, the 3D tissue after printing was removed from the loading platform, the calcium chloride solution was slowly added dropwise onto the 3D tissue until the 3D tissue was just immersed, and then crosslinked for 3 min, the calcium chloride solution was absorbed, and the processed 3D tissue was shown in FIG. 3.

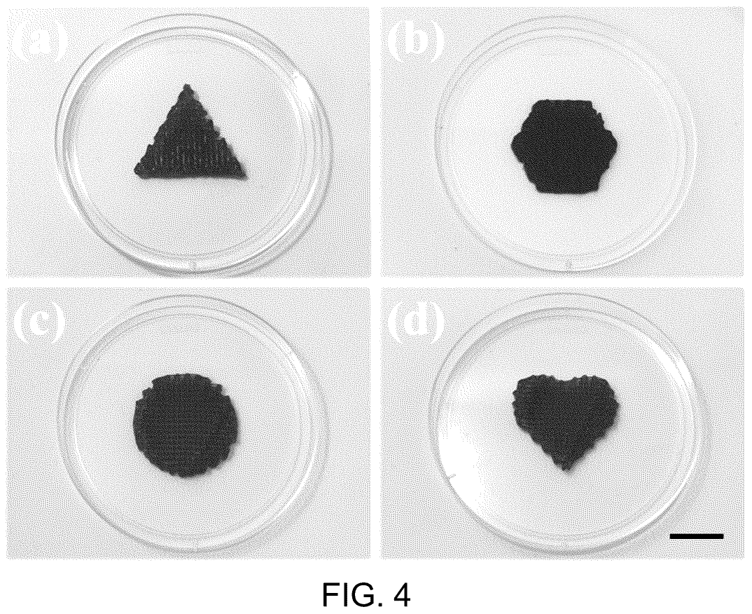

In addition, 3D models of different shapes could be established by using the Auto CAD 2021, and 3D tissue of different shapes such as triangles, hexagons, circles and heart shapes could be obtained after slicing and printing (FIG. 4).

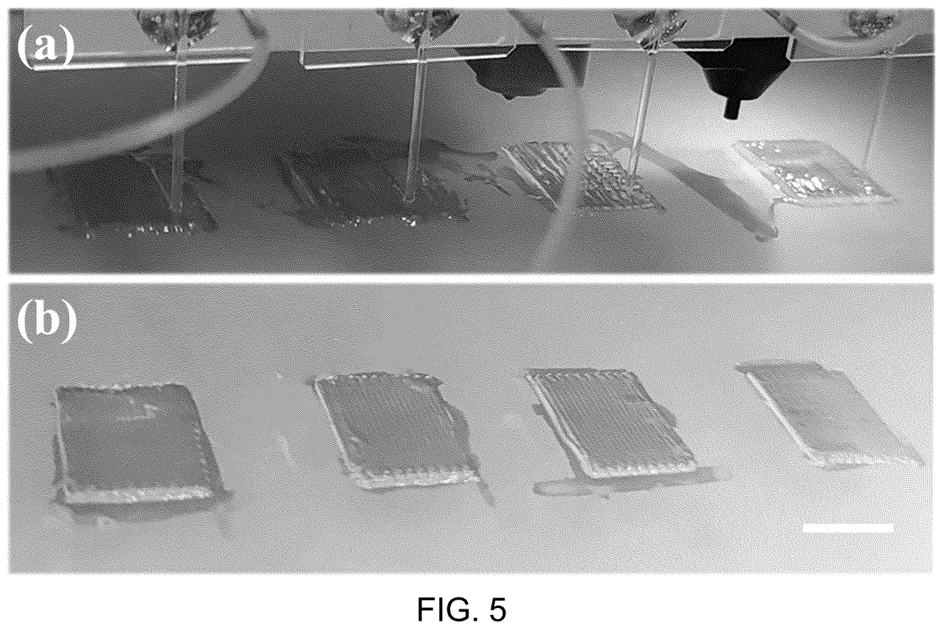

In this example, a plurality of the microfluidic chips could be further installed on the x-axis movable optical axis of the printing moving system 2 by means of magnetic attraction, and multi-nozzle microfluidic 3D printing was performed according to the single-nozzle printing operation, and FIG. 5 illustrated real-time photos and finished product photos of constructing cell-cultured meat by using a multi-nozzle microfluidic 3D printing device integrated with four microfluidic chips.

### Example 4

### Cultivation of 3D Tissue

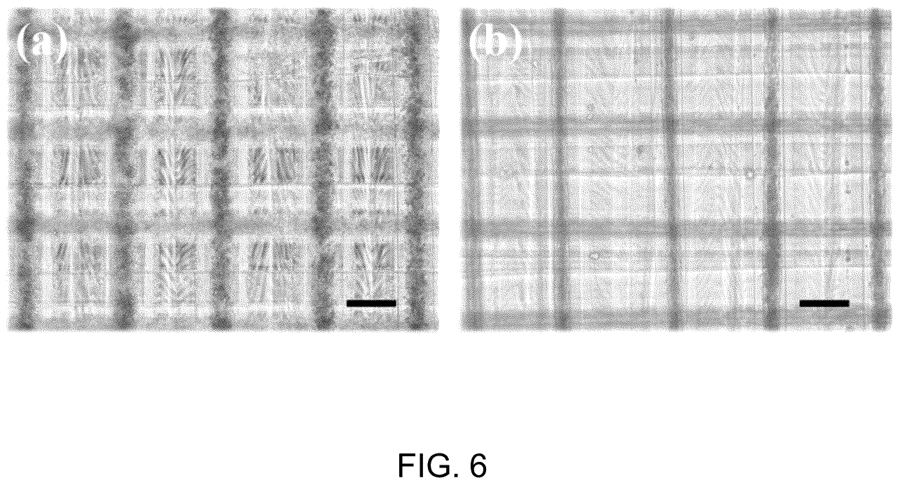

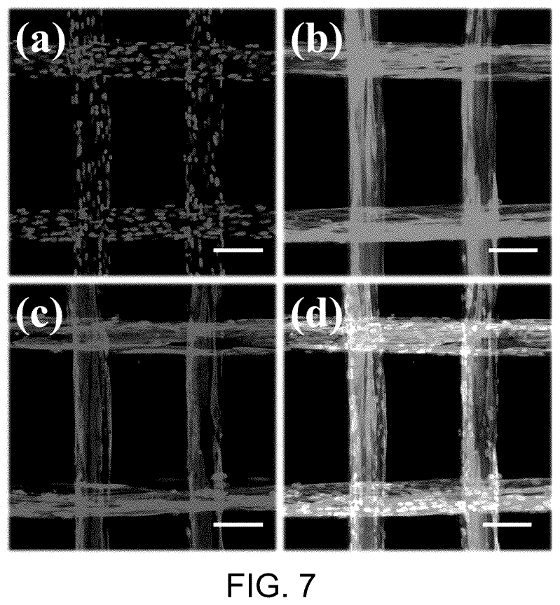

The 3D tissue after final printing and treatment performed in Example 3 was transferred to a culture dish with a diameter of 10 cm containing a culture medium for proliferation culture (DMEM/F-12 with a volume ratio of 84% (Gibco, 11550043), 15% fetal bovine serum (Gibco, 10270-106), 1% penicillin-streptomycin (Gibco, 15140122), containing a final concentration of 5 ng/ml fibroblast growth factor bFGF (R&D, 233-FB-500/CF)) for washing and soaking for 10 min, a volume of the culture medium for proliferation culture in the culture dish was just immersed the 3D tissue, and the 3D tissue was then transferred to an incubator at 37° C. and 5% CO₂ for proliferation culture for 2 d; and
as shown in FIG. 6, the porcine muscle stem cells were observed in a bright field of the microscope, after the porcine muscle stem cells in the 3D tissue were fully migrated and fused to form a fibrous structure, the culture medium for proliferation culture was absorbed, and the 3D tissue was then washed for 2-3 times with a DMEM basal medium without serum. After the washing was completed, a culture medium for differentiation culture (DMEM with a volume ratio of 97% DMEM (C11995500CP, Gibco), 2% horse serum (Hyclone, SH30074.02), and 1% penicillin-streptomycin (Gibco, 15140122)) was added to the culture dish, a volume of the culture medium for differentiation culture in the culture dish was just immersed the 3D tissue, the 3D tissue was then transferred to an incubator at 37° C. and 5% CO₂ for differentiation culture, ½ of the culture medium for differentiation culture in the culture dish was replaced every two days thereafter, and immunofluorescence staining observation was performed after 7 days of differentiation. As shown in FIG. 7, skeleton proteins of the porcine muscle stem cells in the 3D tissue were directionally arranged in a direction of the printed fibers (it could be observed that an F-actin direction was consistent with a direction of the fibers, and seed cells thereof were highly directionally arranged growth), myogenic marker protein myosin was highly expressed, indicating that the seed cells were directionally arranged, migrated and fused to growth in the 3D tissue, and maintained strong myogenic differentiation capability, which was conducive to differentiation and maturation of the seed cells and the synthesis of relevant proteins. Furthermore, a test indicated that the myosin protein in the seed cells was significantly higher than that of a common 2D culture group (the common 2D culture group was a conventional method that directly used porcine muscle stem cells for proliferation and differentiation, and cell usage, proliferation and differentiation culture time, and the like of the porcine muscle stem cells were completely consistent with those of cultivation of the above tissue), further indicating that the differentiation capability of the seed cells was remarkably improved, the synthesis of muscle-related protein was increased, and the production efficiency of the cell-cultured meat was improved. By observing a fiber surface the exposed seed cells and myotube structures could be observed, showing a tissue structure very similar to that of pork skeletal muscle fibers.

### Example 5

### Food Processing of 3D Tissue

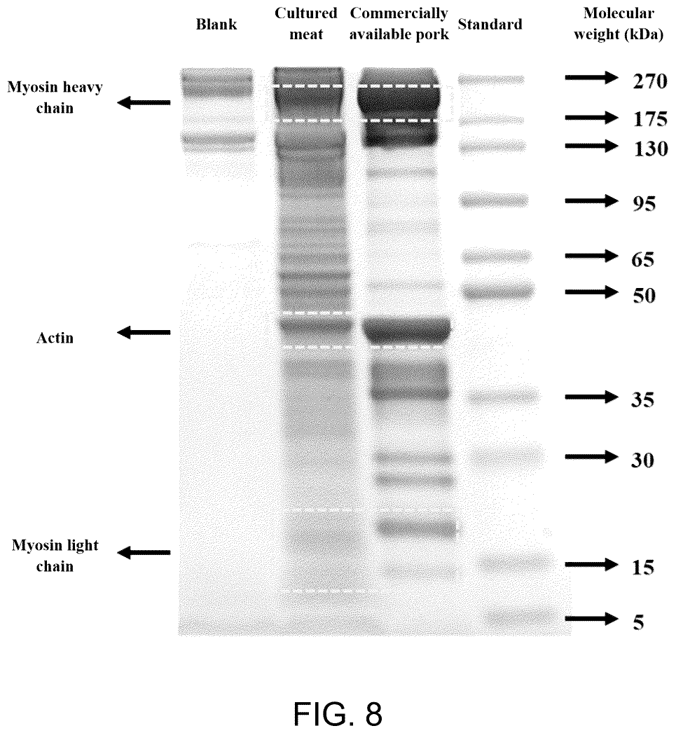

Differentiated mature 3D tissue was harvested and washed with ultrapure water to remove residual culture medium for differentiation culture to obtain preliminary cell-cultured meat, as shown in a gel electrophoresis gel imaging diagram of SDS-PAGE protein in FIG. 8, types and band positions of meat-related proteins (myosin heavy chain, actin and myosin light chain protein, and the like) in the cell-cultured meat are similar to those of commercially available pork; and an amino acid analysis revealed that the content of various amino acids in the cell-cultured meat was not greatly different from that of commercially available pork, especially Gly (glycine), Cys (cysteine) and Pro (proline), which were very close to those of commercially available pork.

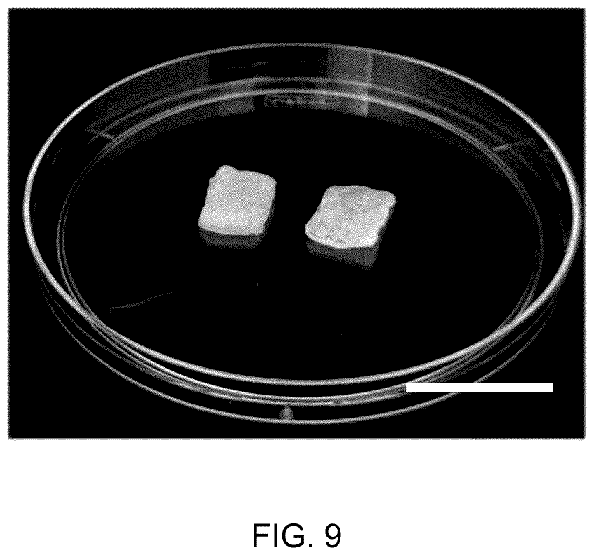

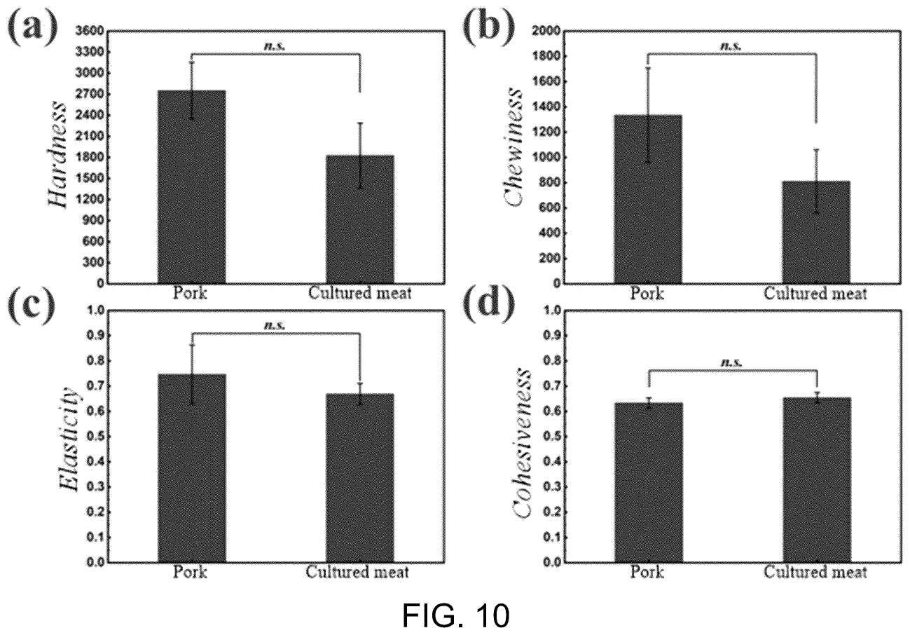

30 mg/mL of sodium alginate solution, 50 mg/mL of gelatin solution and 100 mg/mL of transglutaminase solution were prepared, and 10 mg/mL of calcium chloride solution was prepared for later use; the gelatin solution and the transglutaminase solution were mixed at volume ratio of 9:1 and dripped onto the preliminary cell-cultured meat to fully coat a surface of the preliminary cell-cultured meat, the preliminary cell-cultured meat was then incubated at 37° C. for 2 h, then immersed in the sodium alginate solution for 3 s and fished out, and placed in the calcium chloride solution for crosslinking for 3 min, and the residual calcium chloride solution was then washed and removed to obtain the successfully shaped cell-cultured meat (FIG. 9). After shaping treatment, the cell-cultured meat was subjected to quality analysis, and analysis results indicated that the cell-cultured meat was not significantly different from commercially available pork in terms of four texture indicators, that is, hardness, elasticity, chewiness and cohesiveness (FIG. 10). The cell-cultured meat after shaping was then subjected to food preprocessing (cleaning, seasoning, color enhancement, modeling, sensory quality modification, and the like) and frying to obtain a cell-cultured meat product.

### Example 6

### Cultivation of Microfluidic Biomimetic Fiber

20 mL of F-10 basal culture medium as rinsing liquid was added to a sterile culture dish with a diameter of 10 cm, the three biomimetic fibers of about 20 cm with the “shell-core” structure prepared in Example 3 was clamped with a pair of elbow tweezers at one ends thereof, placed in the rinsing liquid to be washed for 2-3 times to fully remove the residual collection liquid. After being washed, the biomimetic fibers were transferred to a sterile culture dish with a diameter of 10 cm containing a culture medium for proliferation culture (F-10 with a volume ratio of 84% (Gibco, 11550043), 15% fetal bovine serum (Gibco, 10270-106), 1% penicillin-streptomycin (Gibco, 15140122), containing a final concentration of 5 ng/ml fibroblast growth factor bFGF (R&D, 233-FB-500/CF)), and the culture dish was placed in an incubator at 37° C. and 5% CO₂ for proliferation culture for 2 d. the porcine muscle stem cells were observed in a bright field of the microscope, after the porcine muscle stem cells in the biomimetic fibers were fully migrated and fused to form a fibrous structure, the culture medium for proliferation culture was absorbed, and the biomimetic fibers were then washed for 2-3 times with a DMEM basal medium without serum. After the washing was completed, a culture medium for differentiation culture (DMEM with a volume ratio of 97% DMEM (C11995500CP, Gibco), 2% horse serum (Hyclone, SH30074.02), and 1% penicillin-streptomycin (Gibco, 15140122)) was added to the culture dish, which was placed in an incubator at 37° C. and 5% CO₂ for continuous differentiation culture, ½ of the culture medium for differentiation culture in the culture dish was replaced every two days thereafter, and mature biomimetic fibers were obtained after 7 days of differentiation culture.

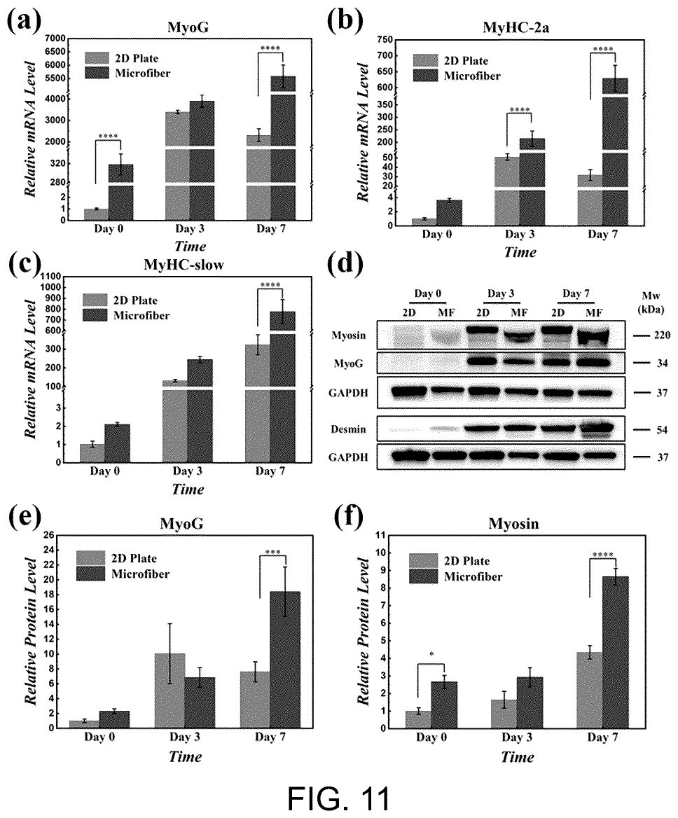

On Day 0, Day 3 and Day 7 of the differentiation, RT-qPCR and Western Blot were used to evaluate changes in gene and protein expression of differentiation of the seed cells cultured in the biomimetic fibers and a 2D culture dish at a molecular biology level, respectively, where the seed cells cultured in the 2D culture dish was a conventional method that directly adopted the porcine muscle stem cells for differentiation, the porcine muscle stem cells were inoculated onto a sterile culture dish with a diameter of 3.5 cm covered with a matrigel for proliferation and differentiation, and cell usage, proliferation and differentiation culture time, and the like, of the porcine muscle stem cells were completely consistent with those of cultivation of the biomimetic fibers. On Day 0, Day 3 and Day 7 of the differentiation, seed cells in the biomimetic fibers and the 2D culture dish were lysed using Trizol, and RNA in the lysed cells was extracted using a total RNA extraction kit for cultured cells of TIANGEN Biotech (Beijing) Co., Ltd.; and after a concentration of the RNA of the sample was determined, the RNA was subjected to reverse transcription by using a reverse transcription kit to obtain cDNA, and a reverse transcription program was set to 37° C. for 15 min and 85° C. for 5 s; the cDNA obtained by reverse transcription was subjected to qPCR reaction by using an RT-qPCR kit, target genes were MyoG, MyHC-2a and MyHC-slow, and a reaction program was set to 95° C. for 30 s, 95° C. for 5 s, and 60° C. for 30 s. As shown in FIGS. 11(a)-(c), expression of the myogenin gene (MyoG) in the seed cells cultured in the biomimetic fibers was over 300 times higher than that of the control group (cultured in the 2D culture dish) at the onset of differentiation (Day 0), and expression of muscle maturation markers, that is myosin synthesis-related genes MyHC-2a and MyHC-slow, in the seed cells cultured in the biomimetic fibers were significantly higher than those of the control group (cultured in the 2D culture dish) at the end of differentiation (Day 7). Further, cell protein samples were obtained by lysing the biomimetic fibers and the cells cultured in the 2D culture dish by using an RIPA lysis buffer, the collected protein samples were centrifuged at 12000 rpm for 5 min at 4° C., supernatant generated therefrom was collected, and a sample protein concentration was measured by using a BCA kit, the sample protein concentration was diluted to 1.25 mg/mL, a quarter of a sample volume of a 5×loading buffer was added, and then mixed evenly to obtain a mixture, and the mixture was heated at 95° C. for 5 min to denature the protein. 20 μL of denatured protein was taken from each sample for SDS-PAGE gel electrophoresis, and the electrophoresis conditions were 80 V for 30 min, and 120 V for 70 min. Then, a PVDF membrane of an appropriate size was cut, the membrane was transferred by rapid wet transfer, a band of a corresponding protein molecular weight (MyHC: 220 kDa; MYOG: 34 kDa; GAPDH: 36 kDa) was cut, and the membrane was blocked with 5% skim milk powder, incubated with a primary antibody at 4° C. overnight, and then incubated with a secondary antibody at room temperature for 2 h; and a solution A and a solution B of a developing solution were mixed at a ratio of 1:1, and then dripped onto the band, which was incubated in the dark for 5 min, the developing solution was then absorbed, an imager was used to develop and pictures were taken, and a grayscale value of the protein band was analyzed by using imageJ software. As shown in FIGS. 11(d)-(f), the differentiation-related protein expression and gene of the seed wells exhibited a same tread. In summary, when the seed wells grew in the biomimetic fibers, differentiation-related genes and protein expressions (MyoG and Myosin protein expression) were significantly higher than those of the 2D culture group. At the onset (Day 0) and end (Day 7) of differentiation, the MyoG protein of the seed cells in the biomimetic fibers were 2.2 times and 2.4 times higher than that of the 2D culture group, respectively; at the onset (Day 0), middle (Day 3) and end (Day 7) of differentiation, the Myosin protein in the biomimetic fibers was 2.66 times, 1.78 times and 2 times higher than that of the 2D culture group, indicating that the differentiation capability of the seed cells was remarkably improved, the synthesis of muscle-related protein was increased, and the production efficiency of the cell-cultured meat was improved.

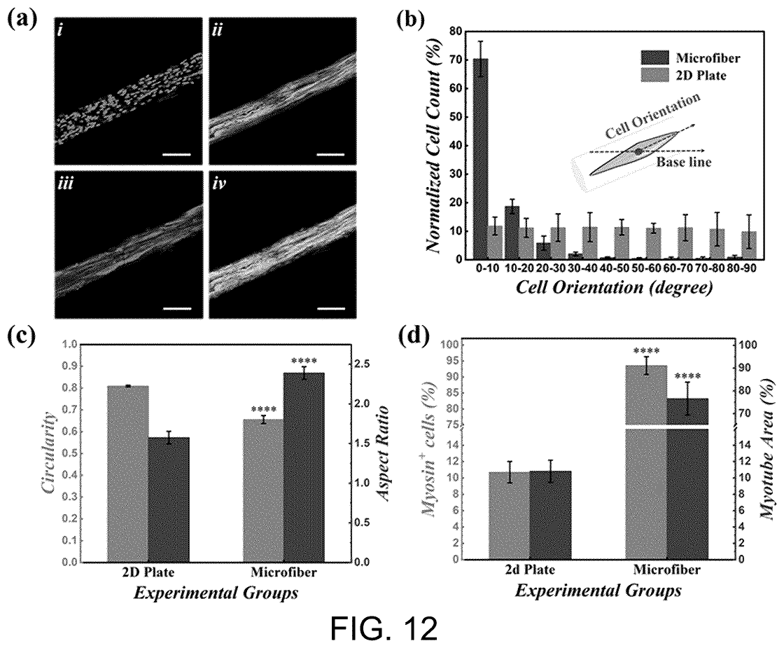

In addition, immunofluorescence staining observation was performed on the biomimetic fibers after 7 days of differentiation. The biomimetic fibers after 7 days of differentiation were fixed by using 4% of paraformaldehyde, the fixed samples were permeabilized with 0.5% Triton X-100 for 30 min, and then blocked with 5% BSA solution for 30 min; the samples were incubated with a primary antibody at 4° C. overnight, and then incubated with a secondary antibody at room temperature for 2 h, and F-actin was further stained with phalloidin for 30 min; and finally, a mounting medium containing DAPI cell nucleus dye was dripped onto the samples for mounting, and the samples were observed and photographed by using a nuclear dye. As shown in FIGS. 12(a)-(d), compared with the 2D culture group, the cytoskeletal proteins in the biomimetic fibers were oriented in a fiber direction (it could be observed that an F-actin direction was consistent with a direction of the fibers, and seed cells thereof were highly oriented growth), and the myogenic marker protein myosin was highly expressed, indicating that the seed cells were oriented, migrated and fused to growth, the differentiation capability of the seed cells was remarkably improved, and the synthesis of muscle-related protein was increased.

1. A cell-cultured meat production device based on microfluidic 3D printing technology, the cell-cultured meat production device comprising a printing nozzle, a printing moving system, a loading platform, a sample injection system and a base; the printing moving system is disposed on the base and is composed of a plurality of movable optical axes, the printing nozzle is fixed on one of the movable optical axes, and the loading platform is connected to another movable optical axis; and the sample injection system is connected to the printing nozzle, and the printing nozzle is a microfluidic chip capable of manipulating, processing, and controlling trace liquid or a sample in a channel,
the cultured meat production device further comprising a biological ink for microfluidic 3D printing, the biological ink is loaded into the sample injection system for printing, the biological ink is a hydrogel solution containing seed cells, or a non-adhesive cell material and the hydrogel solution containing seed cells; the non-adhesive cell material is able to be directly mixed with the hydrogel solution containing seed cells or the non-adhesive cell material encapsulates the hydrogel solution containing seed cells; and the hydrogel solution containing seed cells contains 30%-70% of a biological material and 0.01%-1% of a crosslinking agent in volume ratio, and balance is a basal medium containing a calcium salt and containing the seed cells of 5×10⁶-5×10⁸/mL.

2. The cell-cultured meat production device based on microfluidic 3D printing technology according to claim 1, wherein the printing moving system comprises an x-axis movable optical axis, a z-axis movable optical axis, and a y-axis movable optical axis; the z-axis movable optical axis and the y-axis movable optical axis are fixed on the base, the x-axis movable optical axis is connected to the z-axis movable optical axis, the printing nozzle is fixed on the x-axis movable optical axis, and the loading platform is connected to the y-axis movable optical axis.

3. The cell-cultured meat production device based on microfluidic 3D printing technology according to claim 2, wherein the printing moving system is composed of moving axes capable of driving the printing nozzle to move in two directions on the x-axis movable optical axis and the z-axis movable optical axis, and capable of driving the loading platform to move on the y-axis movable optical axis, and a movable coordinate system configured for the printing moving system is able to be any one of a Cartesian coordinate system, a triangular coordinate system, a polar coordinate system, or a planar joint coordinate system.

4. The cell-cultured meat production device based on microfluidic 3D printing technology according to claim 2, wherein the loading platform has a detachable structure, and is assembled into the printing moving system and connected to the y-axis movable optical axis for assembly line printing; and the loading platform is made of copper, aluminum, iron, steel, alloy, glass, ceramic, or carbon fiber plates.

5. The cell-cultured meat production device based on microfluidic 3D printing technology according to claim 1, wherein the sample injection system comprise a sample loader, a sample injection pump and a pipe, the sample injection pump is placed on a horizontal table top, the sample loader is fixed on the sample injection pump, one end of the pipe is connected to an outlet of the sample loader, the other end of the pipe is connected to an inlet of the printing nozzle, and a feeding manner of the sample injection system comprises piston-type extrusion, pneumatic extrusion or screw-type extrusion.

6. The cell-cultured meat production device based on microfluidic 3D printing technology according to claim 1, wherein a printing control display system and a data transmission system are embedded and installed in the base; and the data transmission system is connected to the printing control display system wirelessly or through a data cable, and the printing control display system is connected to the printing moving system wirelessly or through a data cable.

7. The cell-cultured meat production device based on microfluidic 3D printing technology according to claim 6, wherein the printing control display system is mainly configured to control printing leveling, select a printing program, issue a printing instruction and perform a position adjustment of the printing moving system; the data transmission system is configured to transmit a printing instruction file into a microfluidic 3D printing device; and a data transmission form of the data transmission system comprises USB transmission, memory card transmission or computer transmission.

8. The cell-cultured meat production device based on microfluidic 3D printing technology according to claim 6, wherein after the printing control display system and the data transmission system are embedded in the base, they are powered together with the base.

9. The cell-cultured meat production device based on microfluidic 3D printing technology according to claim 1, wherein the microfluidic chip on the printing moving system is able to be flexibly replaced to perform integrated printing according to different production demands.

10. The cell-cultured meat production device based on microfluidic 3D printing technology according to claim 9, wherein one or more microfluidic chips is able to be integrated on the printing moving system as the printing nozzle, and when a plurality of the microfluidic chips are integrated on the printing moving system, microfluidic chips with a same channel structure or different channel structures is able to be used.

11. The cell-cultured meat production device based on microfluidic 3D printing technology according to claim 1, wherein the microfluidic chip is able to be integrated on the printing moving system by means of clamping, snap-fitting, plugging, magnetic attraction, tenoning, riveting, threaded connection, or bayonet connection.

12. The cell-cultured meat production device based on microfluidic 3D printing technology according to claim 1, wherein materials for making the microfluidic chip comprise one or more of crystalline silicon, polydimethylsiloxane, quartz, polyphthalamide, polymethyl methacrylate, polycarbonate, polystyrene, epoxy resin, acrylic acid, rubber, and fluoroplastic; and a method for making the microfluidic chip comprises glass capillary assembling, machining, etching, or molding.

13. The cell-cultured meat production device based on microfluidic 3D printing technology according to claim 1, wherein the microfluidic chip is able to be of a single-channel type, a coaxial nested type, or a multi-channel parallel type.

14. The cell-cultured meat production device based on microfluidic 3D printing technology according to claim 1, wherein the microfluidic chip is able to, based on microfluidic chips with different structures, generate solid, “shell-core”, hollow, multi-component, spiral, and string-bead fibers for microfluidic 3D printing.

15. A process of preparing cell-cultured meat by use of the cell-cultured meat production device according to claim 1, the process of preparing the cell-cultured meat comprising:
step (1) preparing the biological ink required for microfluidic 3D printing for later use;
step (2) importing a printing instruction file into a microfluidic 3D printing device, loading the biological ink prepared in the step (1) into the sample injection system; connecting the sample injection system to an inlet of the printing nozzle, which is a channel inlet of the microfluidic chip, and squeezing the biological ink into the printing nozzle, which is the microfluidic chip, through the pipe; selecting the instruction file to be printed after fibers are generated at an outlet of the microfluidic chip, and starting the entire microfluidic 3D printing device; and the fibers generated at the outlet of the printing nozzle are deposited on the loading platform driven by the optical axes of the printing moving system, and are stacked and shaped according to a printing instruction path;
step (3) disassembling the loading platform, performing crosslinking and curing treatment on 3D tissue obtained through printing in the step (2), and then transferring the 3D tissue to corresponding culture media for proliferation culture and differentiation culture; and
step (4) harvesting the mature 3D tissue cultured in the step (3), washing to remove the culture medium, and performing food processing to obtain the cell-cultured meat.

16. (canceled)

17. The process of preparing the cell-cultured meat according to claim 15, wherein the non-adhesive cell material in the biological ink is any one or more of sodium alginate, chitosan, pectin, carrageenan, and gellan gum; and a concentration of the non-adhesive cell material solution is 10-50 mg/mL.

18. The process of preparing the cell-cultured meat according to claim 15, wherein the seed cells in the biological ink are derived from any one or more of a pig, cattle, sheep, a chicken, a duck, a rabbit, a fish, a shrimp, and a crab; and the seed cells are one or more of muscle stem cells, myoblasts, myosatellite cells, muscle precursor cells, bone marrow-derived mesenchymal stem cells, adipose-derived mesenchymal stem cells, induced pluripotent stem cells, cardiomyocytes, adipose stem cells, adipose precursor cells, bone marrow-derived adipose cells, fibroblasts, smooth muscle cells, vascular endothelial cells, epithelial cells, neural stem cells, glial cells, osteoblasts, chondrocytes, liver stem cells, hematopoietic stem cells, stromal cells, embryonic stem cells, or bone marrow stem cells.

19. The process of preparing the cell-cultured meat according to claim 15, wherein the biological material is one or more of collagen, recombinant collagen, gelatin, matrigel, hyaluronic acid, silk protein, elastin, spider silk protein, fibrin, fibrinogen, silk fibroin, laminin, fibronectin, integrin, cadherin, nestin, decellularized extracellular matrix, chondroitin sulfate, heparin, keratan sulfate, dermatan sulfate, heparan sulfate, keratin, keratin sulfate, cellulose, polymerase, carboxymethyl cellulose, polylactic acid, polyvinyl alcohol, lecithin, nanocellulose, soy protein, pea protein, gluten protein, rice protein, peanut protein, yeast protein, fungal protein, wheat protein, potato protein, corn protein, chickpea protein, mung bean protein, seaweed protein, almond protein, or quinoa protein.

20. The process of preparing the cell-cultured meat according to claim 15, wherein the basal medium used in the biological ink is one or more of F-10, DMEM, MEM, F-12, DMEM/F-12, DMEM/F-12 GlutamMAX™, F-12K, RPMI 1640, IMDM, L-15, 199, MCDB 131, LHC, or McCoy's 5A.

21. The process of preparing the cell-cultured meat according to claim 15, wherein the crosslinking agent used in the biological ink comprises any one or more of NaOH, KOH, NaHCO₃, HEPES balanced salt solution, EBSS balanced salt solution, HBSS balanced salt solution, PBS, and DPBS, and transglutaminase, tyrosinase, laccase, lysyl oxidase, polyphenol oxidase, catalase, thrombin or genipin.

22. The process of preparing the cell-cultured meat according to claim 15, wherein the hydrogel solution containing seed cells comprises the biological material, the crosslinking agent, and the basal medium containing a calcium salt and the seed cells; each 1 mL of the hydrogel solution contains 290-699 μL of the biological material with a concentration of 4-8 mg/mL, 1-10 μL of the crosslinking agent with a concentration of 1-2 mol/L, and 300-700 μL of the basal medium containing-15-25 mg/mL of the calcium salt with a concentration of 15-25 mg/mol and 1×10⁷-1×10⁸ of the seed cells; the biological material is one or more of the collagen, the recombinant collagen, the gelatin, the matrigel, the hyaluronic acid, or the silk protein; the crosslinking agent comprises one or more of NaOH, KOH, or NaHCO₃; the calcium salt is one or more of calcium chloride, calcium carbonate, calcium sulfate and calcium nitrate; the basal medium is one or more of the F-10, the DMEM, the MEM, the F-12 or the DMEM/F-12; and the seed cells are the muscle stem cells, the myoblasts, the myosatellite cells, or the muscle precursor cell of the pig, the sheep, the chicken or the duck.

23. The process of preparing the cell-cultured meat according to claim 15, wherein the crosslinking and curing treatment in the step (3) preferably comprises one or more of temperature-induced crosslinking, electrostatic interaction crosslinking, ion crosslinking, and enzyme crosslinking.

24. The process of preparing the cell-cultured meat according to claim 15, wherein in the step (3), the culture medium for proliferation culture comprises 79-89% basal medium, 10-20% fetal bovine serum, and 1% penicillin-streptomycin in the volume ratio, containing 1-10 ng/mL alkalic fibroblast growth factor; and the culture medium for differentiation culture comprises 94-97% basal medium, 2-5% horse serum and 1% penicillin-streptomycin in the volume ratio.

25. The process of preparing the cell-cultured meat according to claim 15, wherein the food processing in the step (4) comprises preprocessing and cooking, and the preprocessing comprises one or more of cleaning, seasoning, color enhancement, modeling, sensory quality modification, and the like, and the cooking comprises frying, deep frying, boiling, steaming, baking, and the like.
